(19) United States
(12) Patent Application Publication
(10) Pub. No.: US 2022/0057312 A1
(43) Pub. Date: Feb. 24, 2022

(54) MASS LIQUID FLUIDITY METER AND PROCESS FOR DETERMINING WATER CUT IN HYDROCARBON AND WATER EMULSIONS

(71) Applicant: Cenovus Energy Inc., Calgary (CA)

(72) Inventor: Mark Lawrence FLYNN, Calgary (CA)

(21) Appl. No.: 17/408,896

(22) Filed: Aug. 23, 2021

Related U.S. Application Data
(60) Provisional application No. 63/069,541, filed on Aug. 24, 2020.

(51) Int. Cl. G01N 11/04; G01N 33/28; G01F 1/84; G05D 7/06

(52) U.S. Cl. CPC G01N 11/04; G05D 7/0676; G01F 1/84; G01N 33/2847

(57) ABSTRACT

Disclosed is a process and mass liquid flow (MLF) meter for measuring water cut, the MLF meter including a variable speed pump, a blind mixing T, a Coriolis meter, a differential pressure sensor, and a backpressure valve, and the process including maintaining a velocity of an emulsion through a Coriolis meter within a predetermined threshold from a predetermined velocity by controlling the variable speed pump, maintaining a pressure within the MLF meter to above a minimum pressure by controlling the backpressure valve, measuring a differential pressure across the Coriolis meter utilizing the differential pressure sensor, measuring a mass flow in the MLF meter utilizing the Coriolis meter, determining a viscosity based on the measured differential pressure and mass flow, and determining water cut of the emulsion based on the determined viscosity.

### FIELD

The present disclosure relates to a mass liquid fluidity meter and process for determining water cut in hydrocarbon and water emulsions.

### BACKGROUND

In some hydrocarbon recovery methods, including steam-assisted gravity drainage (SAGD), recovered hydrocarbons are produced as a multiphase emulsion that includes hydrocarbons, water, and gases, such as natural gas. Water cut of multiphase emulsions refers to dispersed phase volume fraction of water within the multiphase emulsion, which is related to the percentage of water within the multiphase emulsion.

Various techniques have been employed to measure water cut in multiphase emulsions. These techniques include utilizing microwave electromagnetic radiation to determine water cut based on the emulsion dielectric constant and conductivity of water, capacitance or conductance measurements to determine water cut based on the dielectric constants variance between oil and water, gamma-ray absorption measurements and utilizing exponential absorption variance between oil and water, infrared absorption measurement and utilizing the absorption variance of infrared energy between oil and water, differential pressure drop or mass flow measurements utilizing the density variance between oil and water, or sampling and laboratory analysis utilizing centrifuge separation of the oil and water components.

These techniques are not suited to measuring across flow spectrum of continuous oil to continuous water in real time or for measuring water cut in emulsions produced in SAGD operations.

For example, the emulsion produced in SAGD operations typically includes bitumen with standard density range from 1000 to 1015 kg/m³ and water with a density range from 999 to 1015 kg/m³. Previous techniques that utilize the density variation between oil and water to determine water cut are not accurate because the density of oil and water in SAGD produced emulsions is too similar for these previous techniques to be effective.

Further, in SAGD operations the steaming of the reservoir leaches salts out of the reservoir and into the produced emulsion. The salt increases the salinity of the water in the emulsion, increasing the density of the water. In the previous techniques for determining water cut, salinity changes must be accounted for, and the relationship to salinity to water cut is not linear. Corrections for salinity changes in the previous techniques require utilizing calibration tables that include various water salinities to water cut biases, with different correction tables being needed over time as salinity changes.

Laboratory analysis utilizing centrifuge separation of oil and gas components is completed offline in by gathering a representative sample of the emulsion. The sample is cooled during sampling to prevent the release of steam during the sampling process and to prevent flashing of any diluent in the emulsion, prior to separation in the centrifuge. The difficulty in safely acquiring samples from flow lines, the delay between acquiring a sample and receiving analysis results, and the costs of performing the analysis make laboratory analysis prohibitive for the purpose for controlling process operations in SAGD operations.

Improvements in determining water cut in multiphase emulsions are desirable.

### BRIEF DESCRIPTION OF THE DRAWINGS

Embodiments of the present disclosure will now be described, by way of example only, with reference to the attached Figures.

FIG. 1 illustrates an example mass liquid fluidity meter according to an embodiment of the present disclosure.

FIG. 2 illustrates another example mass liquid fluidity meter according to another embodiment of the present disclosure.

FIG. 3 illustrates an example control system for a mass liquid fluidity meter according to an embodiment of the present disclosure.

FIG. 4 illustrates an example process for determining water cut of a multiphase emulsion using a mass liquid fluidity meter according to an embodiment of the present disclosure.

FIG. 5 illustrates an example of a blind T used in a mass liquid fluidity meter in accordance with embodiments of the present disclosure.

### DETAILED DESCRIPTION

The present disclosure provides a mass liquid fluidity (MLF) meter and process for determining water cut in multiphase emulsions with increased accuracy over previous meters. Water cut is determined based on a determined viscosity of the multiphase emulsion. The disclosed MLF meter and process determine viscosity of multiphase emulsions with greater accuracy than conventional viscometer by creating uniform emulsion. The disclosed MLF meter determines the emulsion viscosity utilizing a Coriolis meter to determine the mass flow rate and a differential pressure sensor to measure pressure drop.

The measured pressure drop is affected by the viscosity, gas bubble size, water or oil bubble size, flowrate of the emulsion, size of the pipes in the MLF meter, static pressure, and piping configuration. By maintaining the gas bubble size, the water or oil bubble size, the flowrate of the emulsion, the size of the pipes in the MLF meter, the static pressure, and the piping configuration, substantially constant, the measured pressure drop, and measured temperature in cases in which temperature of the emulsion varies, is correlated to the emulsion viscosity, which can be utilized to determine emulsion viscosity with increased accuracy compared to previous methods.

Knowing the relationship of viscosities of the bitumen and water at the flowing temperature allows the MLF to accurately determine the water cut across the range of the meter from 0-100 percent water cut.

In an embodiment, the present disclosure provides a mass liquid fluidity (MLF) meter that includes a variable speed pump at an inlet end of the MLF meter, a blind mixing T joint having an inlet coupled to an outlet of the variable speed pump, a Coriolis meter having an inlet coupled to the outlet of the blind mixing T, a differential pressure sensor having a first connection coupled to the inlet side of the Coriolis meter and a second connection coupled to the outlet side of the Coriolis meter, a backpressure valve at an outlet end of the MLF meter to maintain a static pressure within the MLF meter to above a minimum pressure, and a controller coupled to the variable speed pump and the Coriolis meter and configured to receive a signal from the Coriolis meter indicating measured velocity of an emulsion through the Coriolis meter, determine whether the measured velocity is within a predetermined threshold of a predetermined velocity, in response to determining that the measured velocity is not within the predetermined amount, transmit a signal to the variable speed pump to vary a flow rate of the variable speed pump, wherein the variable speed pump, the blind mixing T, the Coriolis meter, and the backpressure valve are coupled via piping having generally uniform diameters.

In an example embodiment, the controller is configured to receive a signal from the Coriolis meter indicating a measured mass flow of the emulsion through the Coriolis meter, receive a signal from the differential pressure sensor indicating the measured differential pressure, determine, based on the received signals from the Coriolis meter and the differential pressure meter, a viscosity of the emulsion, and based on the determined emulsion, determine a water cut of the emulsion.

In an example embodiment, the MLF meter further includes a temperature sensor connected to the controller and configured to measure the temperature of an emulsion flowing in the MLF meter, and the controller is further configured to receive a signal from the temperature sensor indicating a measured temperature of the emulsion, and determine the viscosity of the emulsion based on the signal from the temperature sensor.

In an example embodiment, a distance between the first and second connection of the differential pressure sensor is determined based on a predetermined water cut range of the MLF meter.

In an example embodiment, a distance between the first and second connection of the differential pressure sensor is determined based on a predetermined temperature fluctuation range of the MLF meter.

In an example embodiment, a measurement uncertainty of the differential pressure sensor substantially matches a measurement uncertainty of the Coriolis meter.

In an example embodiment, the dimensions of the blind mixing T joint are selected based on the predetermined velocity.

In an example embodiment, the minimum pressure is a pressure at which a gas void fraction of an emulsion in the MLF meter is less than 2%.

In an example embodiment, the first and second connections of the differential pressure sensor include capillary transmitter lines.

In an example embodiment, the predetermined threshold is 0.001 m/s.

In another embodiment, the present disclosure provides a process for measuring water cut utilizing a mass liquid fluidity (MLF) meter comprising a variable speed pump, a blind mixing T, a Coriolis meter, a differential pressure sensor, and a backpressure valve, the process includes maintaining a velocity of an emulsion through a Coriolis meter within a predetermined threshold from a predetermined velocity by controlling the variable speed pump, maintaining a pressure within the MLF meter to above a minimum pressure by controlling the backpressure valve, measuring a differential pressure across the Coriolis meter utilizing the differential pressure sensor, measuring a mass flow in the MLF meter utilizing the Coriolis meter, determining a viscosity based on the measured differential pressure and mass flow, and determining water cut of the emulsion based on the determined viscosity.

In an example embodiment, the process includes measuring the temperature of the emulsion in the MLF meter utilizing a temperature sensor included in the MLF meter, wherein determining the viscosity of the emulsion comprising determining the viscosity based on the measured temperature.

In an example embodiment, the minimum pressure is a pressure at which a gas void fraction of an emulsion in the MLF meter is less than 2%.

In an example embodiment, the predetermined threshold is 0.001 m/s.

For simplicity and clarity of illustration, reference numerals may be repeated among the figures to indicate corresponding or analogous elements. Numerous details are set forth to provide an understanding of the embodiments described herein. The embodiments may be practiced without these details. In other instances, well-known methods, procedures, and components have not been described in detail to avoid obscuring the embodiments described.

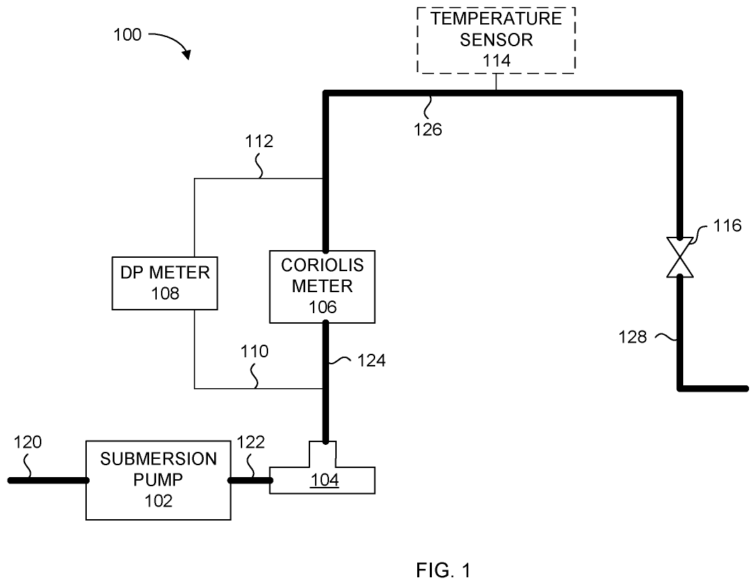

Referring to FIG. 1, a schematic diagram of an example MLF meter 100 according to an embodiment of the present disclosure is shown. The MLF meter 100 includes a submersion pump 102, a blind T 104, a Coriolis meter 106, a differential pressure (DP) meter 108, an optional temperature sensor 114, and a backpressure valve 116. Pipe segments 120-128 couple the various other components of the MLF meter 100 together.

In the example MLF meter 100 shown in FIG. 1, pipe segment 120 forms an inlet of the MLF meter 100 and is coupled to an inlet of the submersion pump 102. The outlet of the submersion pump 102 is coupled to the inlet of the blind T 104 via pipe segment 122. The outlet of the blind T 104 is coupled to the inlet of the Coriolis meter 106 via pipe segment 124. The outlet of the Coriolis meter 106 is coupled to the backpressure valve 116 via pipe segment 126. The DP meter 108 includes a first connection 110 coupled to pipe segment 124 and a second connection 112 coupled to pipe segment 126 in order to measure a pressure differential across the Coriolis meter 106. The outlet of the backpressure valve 116 is coupled to pipe segment 128 which forms the outlet of the MLF meter 100.

The piping segments 120-128 are substantially identical in internal diameter, which is desirable for simplifying the determination of a correction factor, C, utilized for determining emulsion viscosity as described in more detail below.

In an example, the piping segments 120-128 shown in FIG. 1 are arranged to resemble the internal piping loop of the Coriolis meter 106. The internal meter geometry and pipe size diameter of the Coriolis meter 106 may be determined utilizing the manufacturer's specifications. The equation to determine water cut corrects for the meter geometry by using a correction factor, as described in more detail below.

The effect of having the piping segments 120-128 arranged as shown is that the effect of gravity may be ignored when calculating emulsion viscosity, which calculation is described in more detail below with reference to Eq. 1. Further, having piping segments 120-128 arranged as shown in FIG. 1 provides increased accuracy in temperature measurements measured by the optional temperature sensor 114.

In some examples, the DP meter 108 is selected such that the ratio of the range of differential pressure measurable by the DP meter 108 and the smallest gradation of differential pressures measurable by the DP meter 108 is equal to or greater than 2000. The higher the ratio of differential pressure range and the smallest gradation increases the overall accuracy of the water cut determinations of the MLF meter 100. For example, if the range of DP meter 108 is 40-50 kPa, then the smallest graduation size would be determined by 50 kPa/2000, or 0.025 kPa.

The distance between the connections 110, 112 and the DP meter 108 may be determined based on the range of water cut to be measured at the ratio of viscosities of the oil and the water of the flowing emulsion. By increasing the distance between the connections 110, 112 creates an increase in the differential pressure measured by the DP meter 108, for a given water cut. The trade off of increasing the distance between connections 110, 112 and the DP meter 108 is an increase in the piping costs for the MLF meter 100.

In some embodiments, it may be desirable to have the connections 110, 112 of the DP meter 108 as close as possible to the measuring elements of the Coriolis meter 106. This may be accomplished by, for example, tapping the connections 110, 112 into the flanges (not shown) of the Coroilis meter 106, or into the manifold casting (not shown) of the Coriolis meter 106.

In some examples, the connections 110, 112 may be connected to the DP meter 108 via capillary transmitter lines. Capillary transmitter lines may be utilized to isolate the DP sensor from temperature fluctuations in the emulsion or from sand buildup within the piping segments 120-128.

The optional temperature sensor 114 is arranged to measure temperature of the emulsion within the pipe segment 126 in the example MLF meter 100 shown in FIG. 1. In cases in which temperature fluctuates because, for example the temperature is not actively maintained a constant temperature, then the temperature measured by the optional temperature sensor 114 is utilized to determine the viscosity of the emulsion. However, if temperature is actively maintained at a constant temperature, then the optional temperature sensor 114 may be omitted.

In an example, the DP meter 108 and the Coriolis meter 106 have matching measurements uncertainties such that the constant, C, in Eq. 1 set out below is applicable to both the measured differential pressure and the measured mass flow rate. In other examples, the DP meter 108 and the Coriolis meter 106 may have measurement uncertainties, however in this case an additional correction for this deviation may be needed for Eq. 1, set out below.

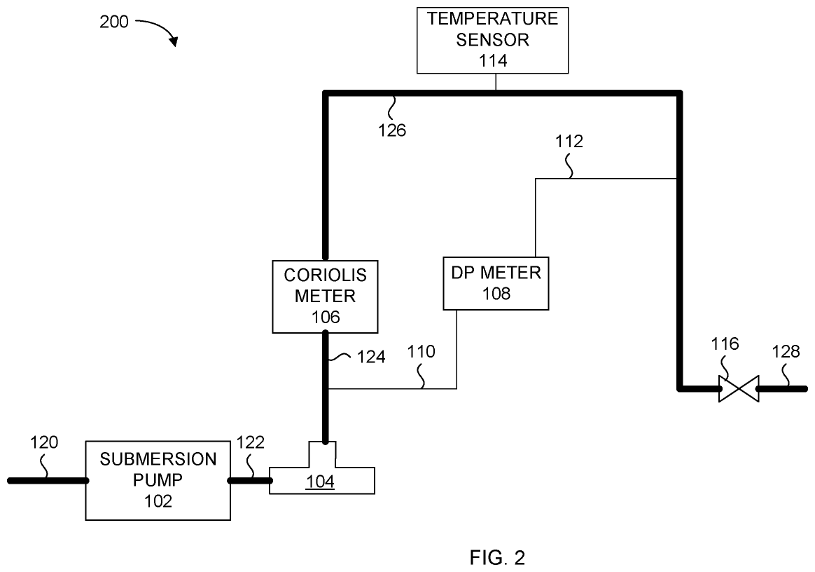

Referring to FIG. 2, an alternative MLF meter 200 is shown in which the arrangement of the connections 110, 112 of the DP meter 108 and the location of the backpressure valve 116 differs from the example MLF meter 100 shown in FIG. 1. The MLF meter 200 may be utilized in situations in which the emulsion's flowing temperature highly fluctuates. Meter 200 may utilize a resistive temperature detector (RTD) as the temperature sensor 114 to measure temperature continuously to correct for fluctuating temperature.

MLF meter 200, the distance between the connections 110, 112 is increased compared to MLF meter 100, and the backpressure valve is moved to a horizontal section of piping section 126, in order to accommodate the temperature sensor 114 being located in between the connections 110, 112 such that the temperature measured by the temperature sensor 114 more accurately reflects the temperature of the emulsion for which viscosity is determined. Although it is previously described that it is desirable to have the connections of the DP meter 100 as close together as possible, in the example MLF meter 200 shown in FIG. 2, there is a tradeoff between having more accurate DP measurements from the DP meter 100 and having a more accurate temperature measurement form the temperature sensor. In cases where the temperature of the emulsion is unstable, the configuration of the connections of the MLF 200 may be desires, whereas in cases in which the temperature of the emulsion is stable, it may be desirable to have the connections of the DP 100 meter closer together.

In operation, a multiphase emulsion is pumped through the MLF meter 100, 200 by submersion pump 102 at a predetermined flow rate. The pipe segment 120 that forms the inlet of the MLF meter 100 may be coupled to, for example, a hydrocarbon well, such as a stream-assisted gravity drainage (SAGD) hydrocarbon well, that is producing a multiphase emulsion. The predetermined flow rate is determined based on the flow rate of the emulsion produced by the SAGD well, such that, for example, the determined flow rate is no greater than 150% and no less than 110%, of the flow rate of the emulsion produced from the SAGD well.

The multiphase emulsions passes through the Coriolis meter, which measures the mass flow rate of the emulsion. The mass flow rate, together with the differential pressure measured by the DP meter 108 are utilized to determined the viscosity of the emulsion, which is then utilized to determine the water cut, as described in more detail below.

Although viscometers for fluids that include a Coriolis meter and a DP sensor have been previously been utilized to estimate the viscosity of fluids, such conventional viscometers do not provide accurate measurements of viscosity in emulsions, particularly multiphase emulsions. The inaccuracy of the estimated viscosity values in conventional viscometers make them unsuitable for determining water cut in multiphase emulsions.

As noted above, by maintaining other factors that affect measured differential pressure, including the gas bubble size, the water or oil bubble size, the flowrate of the emulsion, the size of the pipes in the MLF meter, the static pressure, and the piping configuration, substantially constant, the measured differential pressure is correlated to the emulsion viscosity, and facilitates determining emulsion viscosity values more accurately than previous viscometers.

The present MLF meters 100, 200 are configured to determine viscosity of multiphase emulsions more accurately than conventional viscometers by maintaining substantially constant flow rate through the MLF meter 100, 200, generating an emulsion having uniformly sized bubbles of oil or gas, and by maintaining a static pressure within the MLF meter 100, 200 such that gas in the multiphase emulsion remains in solution and that limits the maximum size of bubbles of water or oil within the emulsion.

The blind T 104 may be utilized to generate substantially uniform sized bubbles of water or oil within the multiphase emulsion, to inhibit phase slip of the multiphase emulsion, or to reduce fluid pulsation in the emulsion that reaches the Coriolis meter 106 due to the submersion pump 102. The viscosity of the emulsion is dependent on the oil or water bubble size within the emulsion, and therefore providing a uniform emulsion is desired to result in a more accurate measurement of emulsion viscosity compared to conventional viscometers.

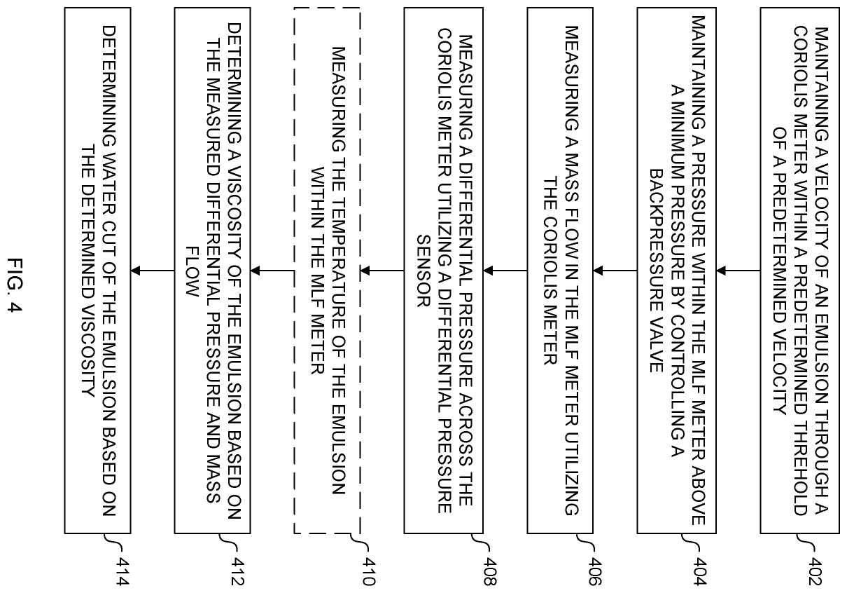

FIG. 5 illustrates how the emulsion fluid mixes within the blind T 104. Fluid from the submersion pump 102 enters into a horizontal section 104a of the blind T, and exits out of a vertical section 104b to the Coriolis meter 106. The horizontal section 104a includes an endwall 500 such fluid from the submersible pump 102 flow into the endwall 500, as illustrated by arrow 501, and bounces back towards the pump 102, as illustrated by arrows 502a-d, causing mixing of the emulsion and reflection of fluid pulsations from the submersible pump 102 back down the horizontal section 104a towards the submersible pump 102.

Passing the multiphase emulsion through the blind T 104 at a predetermined flow rate creates a mixing function in the horizontal section 104a that breaks up the bubbles of water or oil within the emulsion into smaller sized bubbles. The blind T 104 is configured based on the predetermined flow rate of the emulsion through the MLF meter 100, 200 to create uniform bubble sizes at the predetermined flow rate.

The maximum size of the bubbles in the emulsion is determined by the static pressure within the MLF meter 100, 200. The backpressure valve 116 maintains a substantially constant static pressure within the emulsion within the MLF meter 100, 200. The substantially constant static pressure results in a substantially constant maximum bubble size of the emulsion, resulting in greater uniformity of the bubble size compared to varying static pressure. In an example, the backpressure valve 116 is configured to maintain a static pressure of 1000 Kpa. The backpressure valve 116 may be manually controlled by, for example, an operator, or automatically controlled by a controller, as described in more detail below.

Phase slip refers to components of a multiphase mixture separating due to the components travelling at different velocities within a pipe. When a pipe is vertical, such as pipe 124, the difference in the velocities of the different phase components increases compared to travelling horizontally, increasing the phase slip. Sometimes if multiphase fluid velocity is not get enough moving up a vertical pipe phase separation may occur.

By mixing the emulsion in the horizontal section 104a right before the fluid exits the vertical section 104b towards the Coriolis meter 106, as indicated by the arrow 503, phase slip in the emulsion that reaches the Coriolis meter 106 may be reduced than if fluid flowed directly from the submersion pump 102 to the Coriolis meter 106.

To provide emulsion at the Coriolis meter 106 with increased uniformity, it may be desirable to locate the blind T 104 as close Coriolis meter 106 as possible. In an example, the total length from the horizontal section 104a of the blind T 104 to the Coroilis meter 106, i.e., the total length of the vertical section 104b of the blind T 104 and the pipe 124 connecting the blind T 104 to the Coriolis meter 106, is limited to between 3 to 5 times the published inside diameter (PID) of the pipe 124 to reduce phase slippage.

The blind T 104 may also be utilized to reduce the effects of fluid pulsation at the Coriolis meter 106. Fluid pulsation will have a negative effect on the accuracy of the readings of the differential pressure meter 108, and therefore it is desirable to reduce the effects of fluid pulsation as much possible. Pulses in the fluid from the submersion pump 102 are reflected back towards the pump when the fluid hits the endwall 500 of the blind T 104 such that the effects of the fluid pulsation in the fluid that exits vertically through the vertical section 104b are reduced compared to a situation without a blind T 104 in which fluid from the submersion pump 102 flowed directly to the Coriolis meter 106.

Desirably, the design of the blind T 104, particularly the length of the horizontal section 104a is informed by: fluid velocity, water cut, gas void fraction, pressure, and temperature stability. In the case of a MLF meter for use in emulsions produced from SAGD wells, the effectiveness of the blind T 104 for the uses set out above is impacted by changing water cut as: fluid velocity, gas void fraction, temperature, and pressure are stabilized. The length of horizontal section 104a of the blind T 104 is limited to the PID of the horizontal section 104a.

The MLF meters 100, 200 includes a control system that maintains a substantially constant flow rate. The control system also determines the viscosity of the emulsion, and determines the water cut based on the determined viscosity.

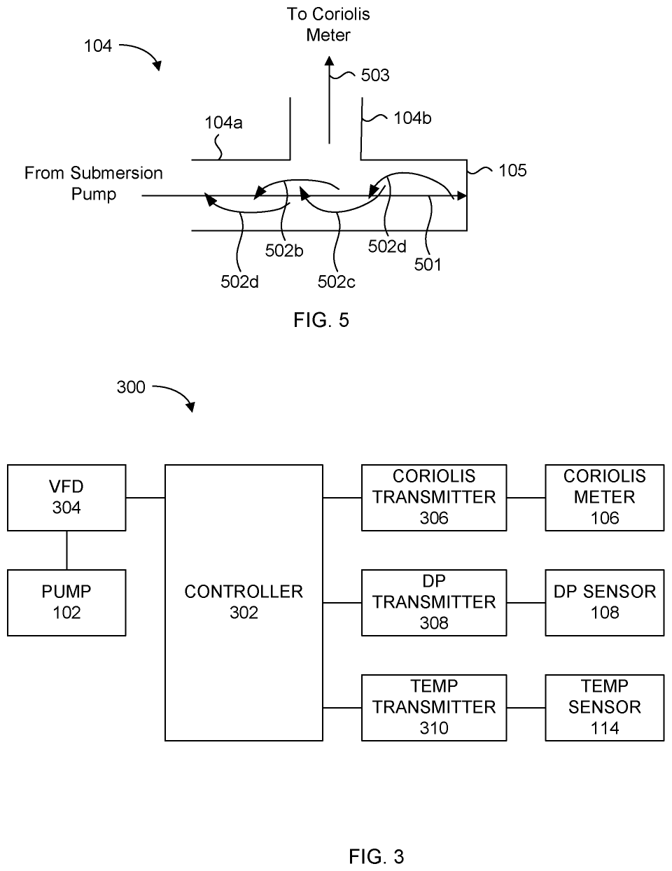

Referring to FIG. 3, a block diagram of an example control system 300 suitable for controlling the example MLF meters 100, 200 is shown. The control system 300 may be utilized for, for example, maintaining the constant flow rate, determining the viscosity of the emulsion, and determining the water cut based on the determined viscosity. The control system 300 includes a controller 302 that controls the overall operation of the MLF meter 100, 200. The controller 302 is in communication with other components of the control system 300 including a variable frequency drive (VFD) 304 that is coupled to the submersion pump 102, a Coriolis transmitter 306 that is coupled to the Coriolis meter 106, a DP transmitter 308 that is coupled to the DP meter 108, and an optional temperature transmitter 310 that is coupled to the temperature sensor 114 is such temperature sensor is included in the MLF meter 100, 200.

The VFD 304 drives the submersible pump 102 at different frequencies to vary the pumping speed, facilitating control over the flow rate of the emulsion through the MLF meter 100, 200. The Coriolis transmitter 306, the DP transmitter 308, and the temperature transmitter 310 transmit signals indicating the measurements taken by the Coriolis meter 106, the DP meter 108, and the temperature sensor 114, respectively.

Although the example control system 300 shown in FIG. 3 shows the VFD 304, the Coriolis transmitter 306, the DP transmitter 308, and the temperature transmitter 310 as being separate components from the submersion pump 102, the Coriolis meter 106, the DP meter 108, and the temperature sensor 114, respectively, in other examples any of the VFD 304, the Coriolis transmitter 306, the DP transmitter 308, or the temperature transmitter 310 may be included within the associated submersion pump 102, Coriolis meter 106, DP meter 108, or temperature sensor 114 as a single component.

In order to provide a substantially constant flow rate, the controller 302 receives a signal from the Coriolis transmitter 306 that indicates a flow rate measured by the Coriolis meter 106. The controller 302 determines whether the measured flow rate is within a predefined threshold amount of a predetermined desired flow rate.

If the measured flow rate is not within predetermined threshold of the predetermined desired flow rate, the controller 302 may vary the flow rate by sending a signal to the VFD 304 to vary the drive frequency provided by the VFD 304 to the submersion pump 102. The flow rate is varied in this manner until the flow rate measured by the Coriolis meter 106 is within the threshold amount of the predetermined flow rate. In an example, the predetermined threshold may be 0.0001 m/s. In other examples, any other suitable manner for varying the flow rate of the emulsion within the MLF meter 100, 200 may be utilized to maintain a flow rate within a threshold amount of a predetermined flow rate.

In examples, the backpressure valve 116 may be coupled to the controller 302 such that the controller 302 controls the backpressure valve 116 to maintain a substantially constant static pressure. For example, a pressure sensor (not shown) may transmit a measured static pressure to the controller 302. If the controller 302 determines that the static pressure is below a minimum pressure, or in some examples, above a maximum pressure, the controller 302 may control the backpressure valve 116 until the static pressure back above the minimum pressure, and in some examples below the maximum pressure.

The controller 302 relies on the variation of viscosity of oil and water to determine the water cut from the viscosity of the emulsion. For example, the viscosity of produced water in the emulsion may be approximately 2.11 cSt and the viscosity of oil in the emulsion may be approximately at 350 cSt at 200° C. Once the overall viscosity of the emulsion is determined, the variation of the viscosities of oil and water is utilized to determine the amount of water, i.e., the water cut, in the emulsion. As set out previously, the viscosity of the emulsion is also affected by the droplet size of the oil or water within the emulsion. By providing a substantially uniform droplet size by maintaining substantially constant flow rate and by passing the emulsion through a blind T and by limiting the maximum droplet size utilizing the backpressure valve, the presently disclosed MLF meters are able to achieve more accurate determination of viscosity than convention viscometers. More accurate determination of viscosity leads to more accurate determination of water cut.

The emulsion viscosity (EV) is determined by the following equation:

$$EV = C \times \frac{DP}{MFR} \qquad ( Eq . 1 )$$

where C is a constant, MFR is the mass flow rate measured by a Coriolis meter, and DP is the differential pressure across the Coriolis meter measured by a DP meter.

The constant C may be referred to as an initial flow constant and is a function of the geometry of the pipe segments of the MLF meter, the distance between the connections of the DP meter, the Coriolis meter size and geometry, the Coriolis meter's stated accuracy, and the DP meter's range and accuracy. In an example, the constant C may be given by the following equation:

C=(πR⁴/8L)×MG (Eq.2)

where R is the radius of the pipe segments of the MLF meter, L is the distance between connections of the MLF meter, and MG is a factor related to the impact of the meter geometry of the particular Coriolis meter, and corrects for installation effects.

The factor MG may be determined through performing a calibration routine on the MLF meter. The calibration routine is performed on start up, i.e., after installation, and may be performed from time to time in response to changes in gas void fraction (GVF) or to changes in water cut (WC). In an example, the following table sets some examples changes in GVF and WC that may make it desirable to perform calibration of the MLF meter, where NA indicates that the MLF meter is not desired for measurements in these ranges:

| On Start | Changes in | Calibration | Changes in | Calibration |
|---|---|---|---|---|
| up | GVF from to | required | WC from to | required |
| Yes | | | | |
| | 0-10 | Yes | 0-10 | NA |
| | 10-15 | Yes | 10-20 | Yes |
| | 15-20 | Yes | 20-30 | Yes |
| | 20-25 | Yes | 30-40 | Yes |
| | 25-30 | Yes | 40-50 | Yes |
| | 30-35 | Yes | 50-80 | Yes |
| | 35- | NA | 80-100 | Yes |

The relationship between emulsion viscosity and the dispersed phase volume fraction of water, referred to as the water cut, is determined based on an empirical relationship. In an example, the following empirical equation based on the Taylors empirical relationship may be utilized to determine water cut:

$${μ}_{emulsion} = {μ}_{oil} ( 1 + x [ 2.5 \times ( \frac{τ + 0.4}{τ + 1} ) ] ) \qquad ( Eq . 3 )$$

where μₒᵢₗ is the viscosity of the oil, τ is the ratio between the water droplets viscosity and the continuous oil phase viscosity, and x is the dispersed phase volume fraction of water, also referred to as the water cut.

The water cut, x, may be determined by setting the emulsion viscosity, EV, determined in Eq. 1, equal to μₑₘᵤₗₛᵢₒₙ in Eq. 2, which gives:

$$EV = {μ}_{oil} ( 1 + x [ 2.5 \times ( \frac{τ + 0.4}{τ + 1} ) ] ) , \\ \frac{EV}{{μ}_{oil}} = 1 + x [ 2.5 \times ( \frac{τ + 0.4}{τ + 1} ) ] , \\ x = ( \frac{EV}{{μ}_{oil}} - 1 ) / [ 2.5 \times ( \frac{τ + 0.4}{τ + 1} ) ] \qquad ( Eq . 4 )$$

Thus, water cut, x, may be calculated utilizing Eq. 4, together with the emulsion viscosity determined by Eq. 1, the known viscosity of the oil, μₒᵢₗ, and the known ratio of the water viscosity to the oil viscosity, τ.

Although the example described above, which results in Eq. 4, is based on utilising the Taylors empirical relationship, any other suitable manner for determining water cut from measured emulsion viscosity may be utilized.

Oil viscosity depends on the amount and type of solvents or diluents, if any, that are present in the emulsion. Solvents and diluents may be included in the emulsion due to solvents or diluents being added into the hydrocarbon formation during production in, for example, a SAGD production process, which solvents and diluents may then flow into the well bore and be produced together with the oil. If solvents and diluents are added over time the oil viscosity may be monitored to accurately determined the water cut, x, from Eq. 4.

The controller 302 may be configured to, for example, receive a concentration of solvents or diluents, or a type of solvent or diluent present in the emulsion, or both the concentration and the type, and determine the oil viscosity, μₒᵢₗ, based on this received information. The controller 302 may determine the oil viscosity, μₒᵢₗ, for given solvent or diluent types and concentrations based on a lookup table stored in, for example, a memory (not shown) of the controller 302 or utilizing a suitable equation for oil viscosity and solvent or diluent concentration.

The viscosity of water will vary based on the type and concentration of impurities that are present in the water. For example, in SAGD operations, the water produced along with the hydrocarbon may be saline, and such salinity may vary over time. Salinity of the water may be accounted for when determining the viscosity of the water, which is used for determining the ratio, τ, in Eq. 4. The variation in viscosity of sea water to fresh water does not need to be correct for within this application, however, to increase accuracy of the MLF this correction maybe applied.

The controller 302 may be configured to, for example, receive a salinity concentration of the water and determine the water viscosity based on the received salinity concentration. The controller 302 may determine the water viscosity for a received salinity concentration utilizing any suitable method including based on a lookup table stored in a memory (not shown) of the controller 302 or utilizing a suitable equation for the relationship between water viscosity and salinity.

As noted above, viscosity the emulsion, oil, and water depends on temperature. In examples in which the emulsion temperature is not maintained at a constant temperature, the controller 302 may be configured to determine the oil viscosity, μₒᵢₗ, based on temperature measurements received from the temperature transmitter 310. The controller 302 may determine the water and oil viscosities for a received temperature utilizing any suitable method including based on a lookup table stored in a memory (not shown) of the controller 302 or utilizing a suitable equation for the relationships between oil and water viscosities and temperature.

In an example, the controller 302 may be further configured to determine oil volume, or standard flow, based on the mass rate measured by the Coriolis meter 106 and the determined water cut. The oil volume may be determined by the following equation:

$$Oil Volume ( \frac{{Sm}^{3}}{hr} ) = \frac{( 1 - X ) \times mass rate ( \frac{kg}{hr} )}{ρ ( \frac{kg}{{m}^{3}} )} \qquad ( Eq . 5 )$$

where ρ is the standard density of oil, and x is the water cut as derived from above equation. The Mass Rate is determined by the Coriolis meter.

Because hydrocarbon reservoir production may be unpredictable, and because the disclosed MLF meters 100, 200 are designed to provide accurate determinations of viscosity and water cut within particular bounds, the controller 302 may be further to configured to provide an alert or alarm that indicate any of operating conditions falling outside of the MLF meter's 100, 200 design criteria, excessive wear of MLF meter 100, 200 components. For example, when break through occurs in a hydrocarbon reservoir, gas or steam slugs, water slugs, sand slugs, or oil slugs may be produced through the well, which may lead to operating conditions that are outside the MLF meter 100, 200 range and/or may damage MLF meter 100, 200 components. The alert or alarm provide an indication to operators of a change in operation conditions or of risk of MLF meter 100, 200 component failure which may affect the accuracy of the determinations made by the MLF meter 100, 200.

In an example, the controller 302 may be configured to detect and provide alerts or alarms for any or all of: low velocity associated with pump failures; high or low differential pressure, which may indicate abnormal viscosity readings; sand buildup in the Coriolis meter; stress failures in Coriolis meter tubing; accuracy self-verification failure in any of the temperature sensor, DP sensor, or Coriolis meter. The alert or alarm may be in any suitable form, and may include audio and/or visual signals, transmitting an electronic message over a network, which message may include information regarding the change in operating conditions or the component at risk of failure, or any other suitable alert or combination of alerts.

Referring now to FIG. 4, a flowchart illustrating a process for measuring water cut utilizing a MLF meter comprising a variable speed pump, a blind mixing T, a Coriolis meter, a differential pressure sensor, and a backpressure valve is shown. The MLF meter may be configured similarly to either of the example MLF meters 100, 200 shown in FIGS. 1 and 2 and described previously. The illustrated process may be carried out by software executed, for example, by a processor of a controller included in a control system incorporated into the MLF meter, such as controller 302 of the control system 300 described previously. Coding of software for carrying out such a process is within the scope of a person of ordinary skill in the art given the present description. The process may contain additional or fewer processes than shown and/or described, and may be performed in a different order. Computer-readable code executable by at least one processor, such as a processor included in the controller 302, to perform the process may be stored in a computer-readable storage medium, such as a non-transitory computer-readable medium.

At 402, a velocity of an emulsion through the Coriolis meter of the MLF meter is maintained within a threshold amount from a predetermined velocity. As disclosed above, the velocity may be maintained at 402 by the controller receiving a measured velocity of the emulsion from the Coriolis meter, and controlling a submersion pump in response to the velocity being more than threshold amount from the predetermined velocity. In an example, the threshold may be 0.001 m/s.

At 404, a static pressure within the MLF meter is maintained to above a minimum pressure by controlling the backpressure valve. As described above, the static pressure is maintained above a minimum pressure such that any gases in the emulsion are within the solution.

The backpressure value is maintained such that oil bubbles or water bubble are maintained within the emulsion and such that the gas bubbles are maintained within solution. The backpressure value that maintains these two conditions is directly related to the maximum operating pressures of the MLF system. The desired backpressure value is also determined by water cut and Gas Void Fraction (GVF) and may be adjusted automatically by adjusting in the drive gain of the Coriolis meter.

The static pressure may be maintained above a minimum pressure, and some examples below a maximum pressure, utilizing a manual back pressure control or by controlling back pressure through, for example, the controller.

At 406, a mass flow of the emulsion is measured utilizing the Coriolis meter. A signal indicating the measured mass flow may be transmitted to the controller by a transmitter associated with the Coriolis meter, as described previously.

At 408, a differential pressure of the emulsion is measured across the Coriolis meter utilizing the differential pressure sensor. A signal indicating the measured differential pressure may be transmitted to the controller by a transmitter associated with the differential pressure sensor, as described previously.

Optionally at 410, a temperature of the emulsion in the MLF meter is measured utilizing a temperature sensor. A signal indicating the measured temperature may be transmitted to the controller by a transmitter associated with the temperature sensor. As described previously, the temperature may be measured 410 when the temperature of the emulsion in the MLF meter fluctuates. For example, if the temperature is not actively maintained at a set temperature, then temperature may be measured at 410.

It may be desired that the measurements at 406, 408, and 410 be taken at approximately the same time to ensure that the measurements correspond temporally to each other.

At 412, the viscosity of the emulsion is determined based on the measured differential pressure, the measured mass flow, and if temperature is measured at 410, the measured temperature. The viscosity of the emulsion may be determined by the controller, such as controller 302, utilizing Eq. 1 and Eq. 2 as previously described in detail.

At 414, the water cut of the emulsion is determined based on the viscosity that is determined at 412. The water cut may be determined utilizing Eq. 4, as previously described in detail. In other examples, the water cut is determined utilizing any other suitable relationship between the viscosity of a multiphase emulsion and the water cut. The determination of water cut at 414 may be based on the type and concentration of solvents or diluents present in the emulsion, the salinity of the water within the emulsion, and the measured temperature of the emulsion in cases in which the temperature is measured at 410, as described in detail above.

Embodiments of the present disclosure provide MLF meters and processes for determining water cut in multiphase emulsions of oil and water. The MLF meters according to the present disclosure measure the emulsion water cut from continuous oil to continuous water utilizing off the shelf devices, configured to achieve accurate and repeatable results for water cut of a SAGD emulsion (a mixture of Bitumen, water, steam, sand, and natural gases).

The disclosed MLF meters and processes operate by determining viscosity of the emulsion, and water cut of the emulsion based on the determined viscosity. The accuracy of the viscosity determination of the disclosed MLF meter and process is improved over conventional viscometers by maintaining factors other than viscosity that affect measured differential pressure, including the gas bubble size, the water or oil bubble size, the flowrate of the emulsion, the size of the pipes in the MLF meter, the static pressure, and the piping configuration, substantially constant such that the measured differential pressure is correlated to the emulsion viscosity.

The substantially constant flow rate is created by utilizing a controller to control the speed of a pump of the MLF meter based on the flow rate measured by the Coriolis meter. The uniform bubble size is generated by passing the emulsion through a blind T, which is configured based on the predetermined flow rate to create a mixing function that breaks up the bubbles of water or oil within the emulsion into smaller sized bubbles, and by utilizing a backpressure valve to maintain a static pressure in the MLF meter above a minimum pressure that sets a maximum bubble size for the emulsion. The minimum static pressure is selected such that, for example, substantially all, for example less than 2 percent, of the gas in the multiphase emulsion remains in solution, which also increases the accuracy of the viscosity determination compared with conventional viscometers.

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

Embodiments of the disclosure can be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device, and can interface with circuitry to perform the described tasks.

The above-described embodiments are intended to be examples only. Alterations, modifications and variations can be effected to the particular embodiments by those of skill in the art without departing from the scope, which is defined solely by the claims appended hereto.

What is claimed is:

1. A mass liquid fluidity (MLF) meter comprising:
a variable speed pump at an inlet end of the MLF meter;
a blind mixing T joint having an inlet coupled to an outlet of the variable speed pump;
a Coriolis meter having an inlet coupled to the outlet of the blind mixing T;
a differential pressure sensor having a first connection coupled to the inlet side of the Coriolis meter and a second connection coupled to the outlet side of the Coriolis meter;
a backpressure valve at an outlet end of the MLF meter to maintain a static pressure within the MLF meter to above a minimum pressure; and
a controller coupled to the variable speed pump and the Coriolis meter and configured to:
receive a signal from the Coriolis meter indicating measured velocity of an emulsion through the Coriolis meter;
determine whether the measured velocity is within a predetermined threshold of a predetermined velocity;
in response to determining that the measured velocity is not within the predetermined amount, transmit a signal to the variable speed pump to vary a flow rate of the variable speed pump;
wherein the variable speed pump, the blind mixing T, the Coriolis meter, and the backpressure valve are coupled via piping having generally uniform diameters.

2. The MLF meter of claim 1, wherein the controller is configured to:
receive a signal from the Coriolis meter indicating a measured mass flow of the emulsion through the Coriolis meter;
receive a signal from the differential pressure sensor indicating the measured differential pressure;
determine, based on the received signals from the Coriolis meter and the differential pressure meter, a viscosity of the emulsion; and
based on the determined emulsion, determine a water cut of the emulsion.

3. The MLF meter of claim 1, further comprising a temperature sensor connected to the controller and configured to measure the temperature of an emulsion flowing in the MLF meter, and wherein the controller is further configured to:
receive a signal from the temperature sensor indicating a measured temperature of the emulsion; and
determine the viscosity of the emulsion based on the signal from the temperature sensor.

4. The MLF meter according to claim 1, wherein a distance between the first and second connection of the differential pressure sensor is determined based on a predetermined water cut range of the MLF meter.

5. The MLF meter according to claim 1, wherein a distance between the first and second connection of the differential pressure sensor is determined based on a predetermined temperature fluctuation range of the MLF meter.

6. The MLF meter according to claim 1, wherein a measurement uncertainty of the differential pressure sensor substantially matches a measurement uncertainty of the Coriolis meter.

7. The MLF meter according to claim 1, wherein the dimensions of the blind mixing T joint are selected based on the predetermined velocity.

8. The MLF meter according to claim 1, wherein the minimum pressure is a pressure at which a gas void fraction of an emulsion in the MLF meter is less than 2%.

9. The MLF meter according to claim 1, wherein the first and second connections of the differential pressure sensor include capillary transmitter lines.

10. The MLF meter according to claim 1, wherein the predetermined threshold is 0.001 m/s.

11. A process for measuring water cut utilizing a mass liquid fluidity (MLF) meter comprising a variable speed pump, a blind mixing T, a Coriolis meter, a differential pressure sensor, and a backpressure valve, the process comprising:
maintaining a velocity of an emulsion through a Coriolis meter within a predetermined threshold from a predetermined velocity by controlling the variable speed pump;
maintaining a pressure within the MLF meter to above a minimum pressure by controlling the backpressure valve;
measuring a differential pressure across the Coriolis meter utilizing the differential pressure sensor;
measuring a mass flow in the MLF meter utilizing the Coriolis meter;
determining a viscosity based on the measured differential pressure and mass flow; and
determining water cut of the emulsion based on the determined viscosity.

12. The process of claim 11, further comprising measuring the temperature of the emulsion in the MLF meter utilizing a temperature sensor included in the MLF meter, wherein determining the viscosity of the emulsion comprising determining the viscosity based on the measured temperature.

13. The process of claim 11, wherein the minimum pressure is a pressure at which a gas void fraction of an emulsion in the MLF meter is less than 2%.

14. The process of claim 11, wherein the predetermined threshold is 0.001 m/s.
